(19) United States
(12) Patent Application Publication
(10) Pub. No.: US 2012/0184816 A1
(43) Pub. Date: Jul. 19, 2012

(54) GUIDE ASSEMBLY FOR ENDOSCOPE

(76) Inventors: Tsuyoshi ASHIDA, Kanagawa (JP); Takayuki NAKAMURA, Kanagawa (JP); Shinichi YAMAKAWA, Kanagawa (JP); Yasunori OHTA, Kanagawa (JP); Masayuki IWASAKA, Kanagawa (JP)

(21) Appl. No.: 13/341,677

(22) Filed: Dec. 30, 2011

(30) Foreign Application Priority Data
Jan. 13, 2011 (JP) 2011-004994

(51) Int. Cl. A61B 1/00

(52) U.S. Cl. 600/114

(57) ABSTRACT

An endoscope has an elongated tube for entry in a body cavity. A guide assembly includes a shaft sleeve for mounting on the elongated tube. A housing sleeve is supported around the shaft sleeve. Three endless belts are secured to the housing sleeve, for endlessly moving in an axial direction of the elongated tube, and propelling the elongated tube by contacting a cavity wall of the body cavity. A driving device is contained in the housing sleeve, for driving the endless belts. Three curved walls constitute the housing sleeve, and support the endless belts movably and in a transversely curved state. Each of the endless belts includes an upper belt run, curved by the curved walls convexly outwards, for contacting the body cavity. A lower belt run is disposed inside, and tensioned between the driving device and the belt rollers.

### BACKGROUND OF THE INVENTION

1. Field of the Invention

The present invention relates to a guide assembly for an endoscope. More particularly, the present invention relates to a guide assembly for propelling an elongated tube of an endoscope into a body cavity, and in which a cavity wall of a body cavity accessed by the endoscope can be protected even in propulsion of the guide assembly.

2. Description Related to the Prior Art

A diagnosis by use of an endoscope is well-known in the field of the medicine. The endoscope includes a head assembly with a CCD or other image sensor, and a section of an elongated tube where the head assembly is disposed at its distal end. The elongated tube is entered in a body of a patient. An image is obtained by the image sensor. A display panel is driven to display the image, for imaging of an object in the body.

A guide assembly for use with the endoscope by assisting the entry in a body cavity has been suggested recently. U.S. Pat. Ser. No. 2005/0272976 (corresponding to JP-A 2005-253892) discloses the guide assembly including a sleeve for mounting on the elongated tube of the endoscope, and endless belts secured to the sleeve in a circulating manner. An outer surface of the endless belts is set in contact with a cavity wall of a body cavity or gastrointestinal tract, before the endless belts are turned around to propel the endoscope into the body cavity by use of friction with a cavity wall of the body cavity. This is effective in facilitating entry of the endoscope into the body cavity such as a large intestine which is an organ of a highly tortuous shape, specifically for an unskilled medical service provider in relation of manipulating the endoscope.

In U.S. Pat. Ser. No. 2005/0272976, an upper belt run of the endless belts is nearly flat. As a shape of the body cavity or gastrointestinal tract is nearly circular as viewed in a cross section, the lateral side portions of the endless belts are likely to contact the cavity wall of the body cavity without a contact of its center portion in a transverse direction with the cavity wall. There is a problem of occurrence of entanglement of the cavity wall of the body cavity in an area of an inner surface of the endless belts for damaging upon the contact of the lateral side portions with the cavity wall of the body cavity.

### SUMMARY OF THE INVENTION

In view of the foregoing problems, an object of the present invention is to provide a guide assembly for propelling an elongated tube of an endoscope into a body cavity, and in which a cavity wall of a body cavity accessed by the endoscope can be protected even in propulsion of the guide assembly.

In order to achieve the above and other objects and advantages of this invention, a guide assembly for an endoscope including a section of an elongated tube for entry in a body cavity is provided. There is a shaft sleeve for mounting on the elongated tube. A housing sleeve is disposed around the shaft sleeve. Plural endless belts are secured to the housing sleeve, for endlessly moving in an axial direction of the elongated tube, and propelling the elongated tube by contacting a cavity wall of the body cavity. A driving device is contained in the housing sleeve, for driving the endless belts. An outer support device is disposed with the housing sleeve, for supporting the endless belts in a transversely curved state to raise a center portion thereof outwards.

Furthermore, plural belt rollers are secured to the housing sleeve in a rotatable manner, for pressing each of the endless belts for contact with the driving device. A contact area of the endless belts in contact with the belt rollers has higher rigidity than a remaining area thereof.

The belt rollers contact the contact area of the endless belts at a center portion thereof in a transverse direction.

Furthermore, a belt ridge is formed to project from an inner surface of the endless belts.

The housing sleeve is in a shape of a polygonal prism, and the outer support device is disposed on each of side edges of the polygonal prism.

The outer support device includes a pair of curved walls, arranged along one another, for placement of longitudinal portions of the endless belts.

The polygonal prism is a triangular prism.

Preferably, the housing sleeve is cylindrical.

The outer support device is constituted by a plurality of outer support devices arranged in an angularly equidistant form around the housing sleeve, and each of the outer support devices includes a pair of curved walls, disposed along one another, for placement of longitudinal portions of the endless belts.

Preferably, furthermore, plural belt rollers are secured to the housing sleeve in a rotatable manner, for contacting each of the endless belts in a circulating manner in the axial direction. A portion of the endless belts in contact with the belt rollers has higher rigidity than a remaining portion thereof.

The belt rollers have a length smaller than a belt width of the endless belts in a transverse direction thereof.

Each of the endless belts includes an upper belt run, curved by the curved wall convexly outwards, for contacting the body cavity. A lower belt run is disposed inside and along the upper belt run, and tensioned between the driving device and the belt rollers.

The driving device includes a drive sleeve, contained in the housing sleeve, and supported around the shaft sleeve in a rotatable manner. Worm gear teeth formed around the drive sleeve. An engagement roller has gear teeth meshed with the worm gear teeth, for turning around the endless belts by engagement therewith.

The belt rollers are offset from the engagement roller in the axial direction, and apply tension to the endless belts engaged with the engagement roller toward the drive sleeve.

The engagement roller has a first roller surface, curved in a transverse direction of the endless belts, for curving the lower belt run in the transverse direction.

The belt roller has a second roller surface having a roller diameter changing according to the first roller surface, for receiving the lower belt run.

The first roller surface is convex and curves the lower belt run convexly outwards.

The belt roller has a second roller surface having a roller diameter decreasing at a roller center portion, for receiving the lower belt run driven by the first roller surface.

Corners of lateral side portions of the endless belts are rounded off.

The curved wall is constituted by plural curved walls. The housing sleeve includes plural substantially flat side walls disposed alternately with the curved walls and in an arrangement shape of a polygonal prism.

The polygonal prism is a triangular prism.

In one preferred embodiment, the housing sleeve is cylindrical.

The housing sleeve includes a cylindrical peripheral surface, curved with a smaller radius than a radius of an outer surface of the curved wall with respect to a sleeve axis. An erect surface is disposed erectly from the cylindrical peripheral surface to the outer surface of the curved wall.

Accordingly, a cavity wall of a body cavity accessed by the endoscope can be protected from interference of endless belts even in propulsion of the guide assembly, because the side rail portions can operate for regulating the lateral side portions of the endless belts.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope;

FIG. 2 is a perspective view illustrating a guide assembly;

FIG. 3 is an exploded perspective view illustrating the guide assembly;

FIG. 4 is a vertical section illustrating the guide assembly;

FIG. 5 is a cross section illustrating components of the guide assembly including belt rollers;

FIG. 6 is a cross section illustrating components of the guide assembly including engagement rollers;

FIG. 7 is a perspective view illustrating one preferred guide assembly of which a housing sleeve is cylindrical;

FIG. 8 is an exploded perspective view illustrating the guide assembly;

FIG. 9 is a cross section illustrating components of the guide assembly including belt rollers;

FIG. 10 is a cross section illustrating components of the guide assembly including engagement rollers.

### DETAILED DESCRIPTION OF THE PREFERRED EMBODIMENT(S) OF THE PRESENT INVENTION

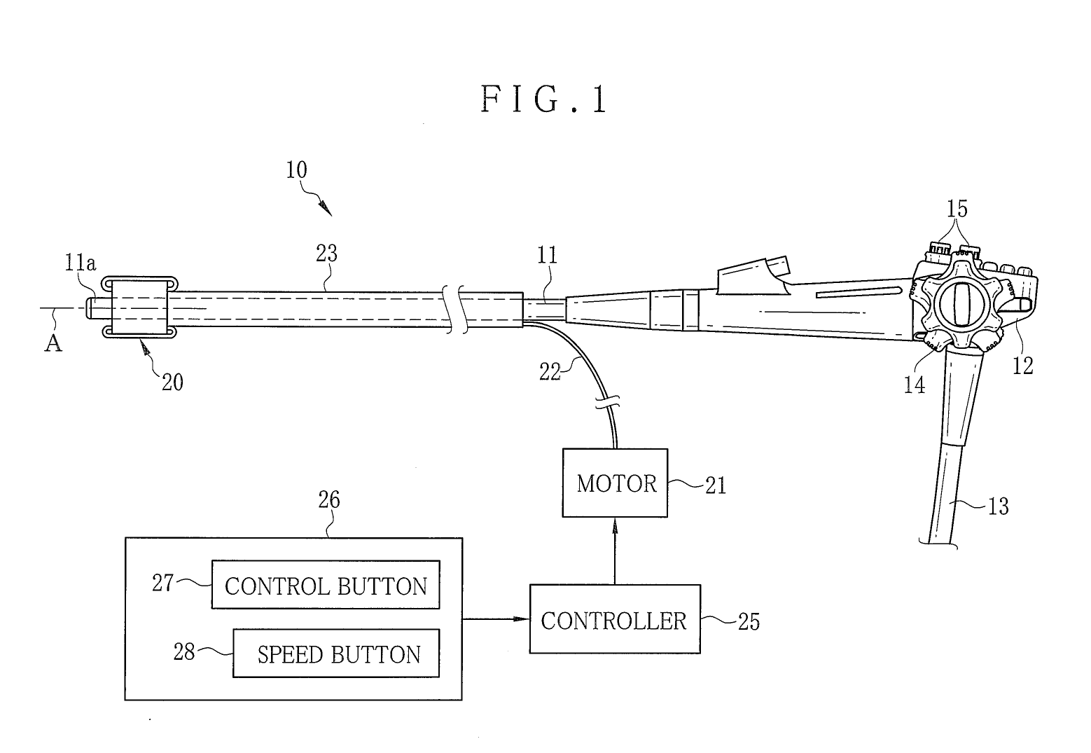

In FIG. 1, an endoscope 10 includes a section of an elongated tube 11 or guide tube, a handle device 12 and a universal cable 13. The elongated tube 11 has the image pickup device in a head assembly 11a, such as a CCD sensor or CMOS sensor of a micro size. The handle device 12 is used for grasping the endoscope 10 and operating the elongated tube 11. The universal cable 13 connects the endoscope 10 to a processing apparatus, a light source apparatus (not shown) and the like.

The elongated tube 11 is a flexible tube of a great length. The head assembly 11a is disposed at a distal end of the elongated tube 11. Various openings are formed in an end surface of the head assembly 11a, including an imaging window, lighting window, end nozzle (all not shown) and the like. The handle device 12 includes steering wheels 14 and flow control buttons 15. The steering wheels 14 are rotated to change a direction and an amount of steering. The flow control buttons 15 are used for supply of air or water and for suction.

The universal cable 13 is connected to the handle device 12. The universal cable 13 includes a signal cable, a light guide device, and a flow channel. The signal cable transmits an image signal from the image pickup device. The flow channel is formed to supply air or water to the head assembly 11a.

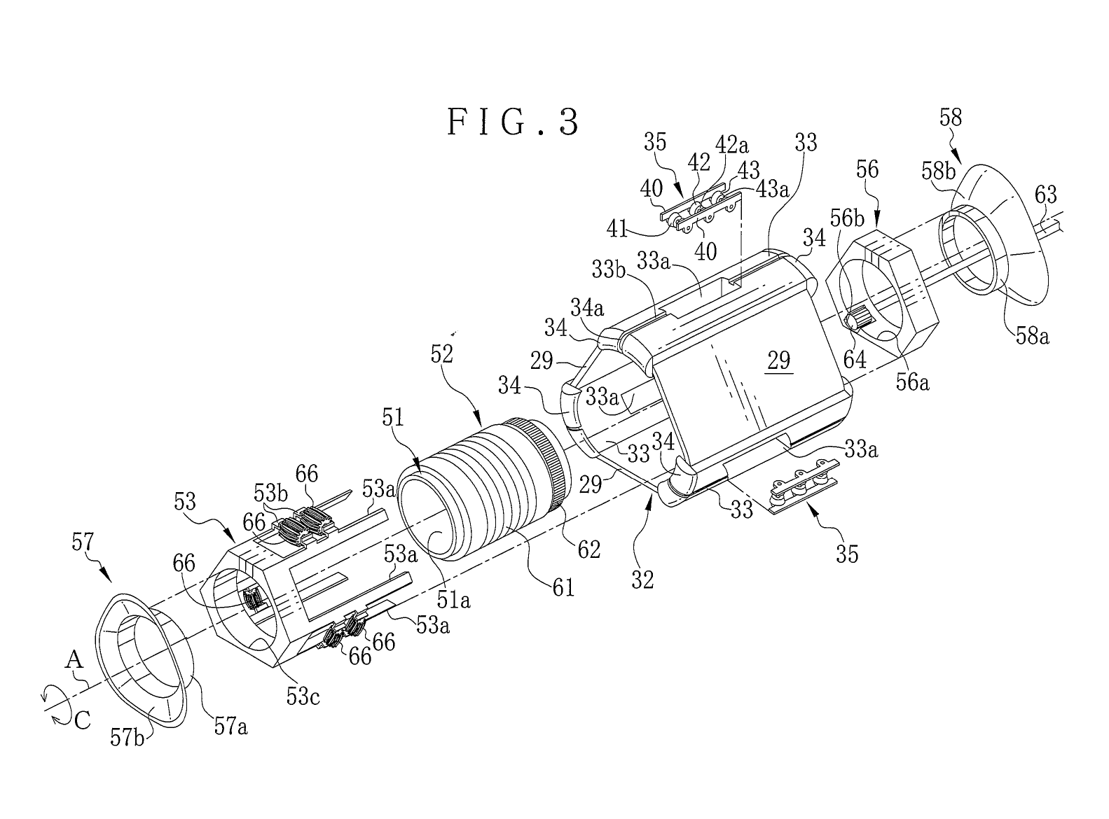

A guide assembly 20 is mounted on the head assembly 11a of the elongated tube 11 in a removable manner, and causes the elongated tube 11 to move in proximal and distal directions in the body cavity. A motor 21 drives the guide assembly 20. A coil structure 63 or torque wire is connected to an output shaft of the motor 21, and transmits torque to rotate for propulsion of the guide assembly 20. See FIG. 3. A protection sheath 22 covers the whole of the coil structure 63. The coil structure 63 rotates in the protection sheath 22 when the motor 21 rotates.

A controller 25 controls the motor 21. An input panel 26 or user interface is connected to the controller 25. The input panel 26 includes a control button 27 and a speed button 28. The control button 27 is operable for inputting command signals for advance, return and stop of the guide assembly 20. The speed button 28 is operable for changing a speed of propulsion of the guide assembly 20.

An overtube 23 is used, in which the elongated tube 11 is mounted. The protection sheath 22 is disposed between the overtube 23 and the elongated tube 11 to extend through.

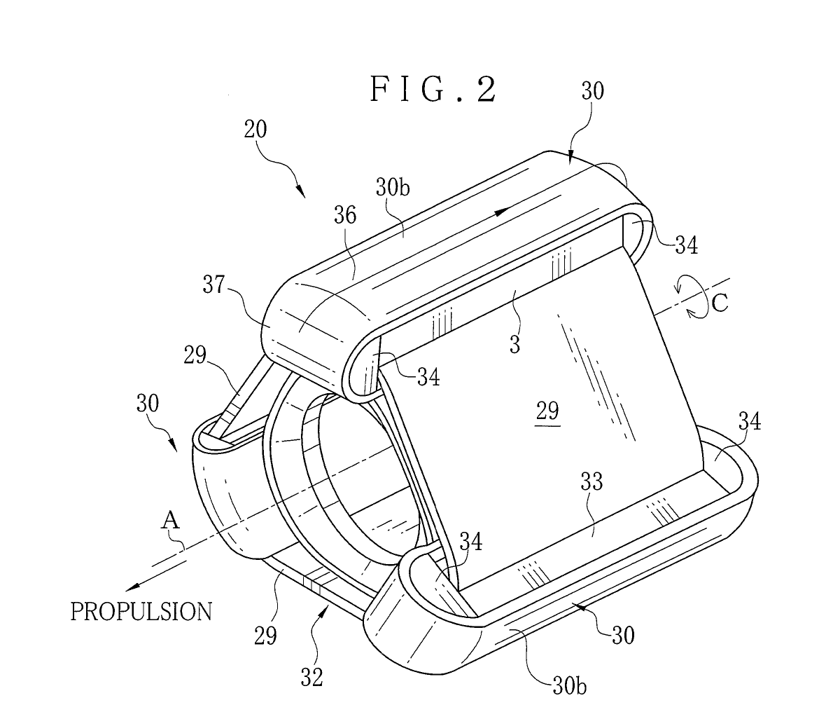

In FIG. 2, plural endless belts 30 are disposed in the guide assembly 20, contact a cavity wall of the gastrointestinal tract, and apply force to the cavity wall in a proximal direction which is reverse to the distal direction of the elongated tube 11 according to an axial direction A of the endoscope 10. A housing sleeve 32 supports the endless belts 30 to circulate in the axial direction A. The endless belts 30 are arranged rotationally equidistantly in a rotational direction C defined about the axis of the axial direction A. An example of the number of the endless belts 30 is three. An outer surface 30b of an upper belt run of the endless belts 30 contacts a cavity wall of the gastrointestinal tract. The arrow in FIG. 2 indicates a direction of the circulation. A material for forming the endless belts 30 is biocompatible plastic material having flexibility, such as polyvinyl chloride (PVC), polyamide resin, fluorocarbon resin, polyurethane and the like.

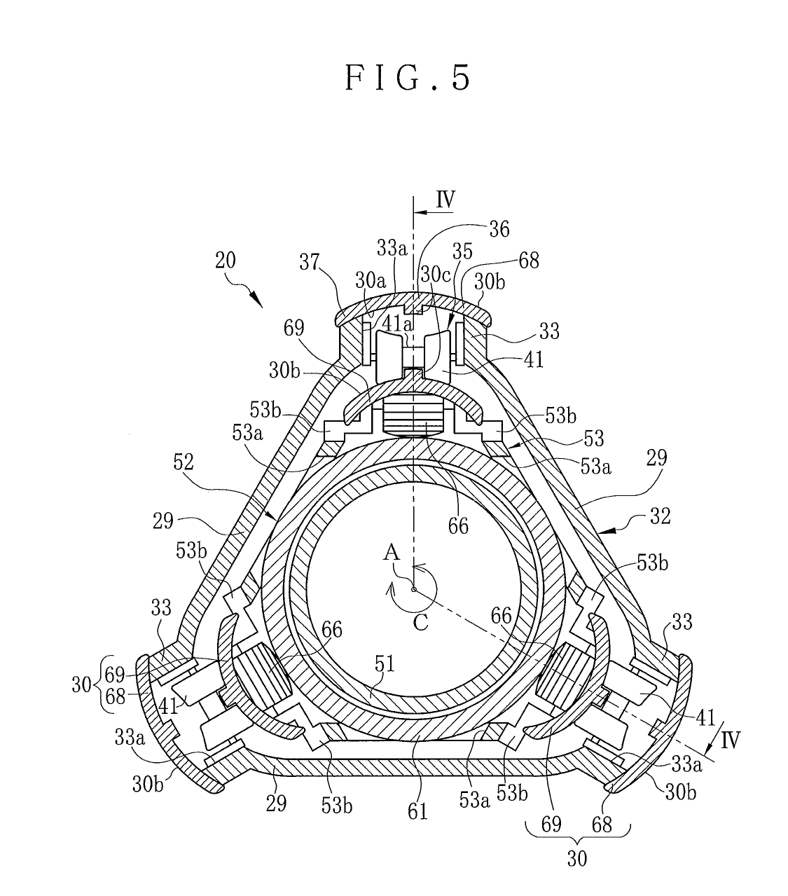

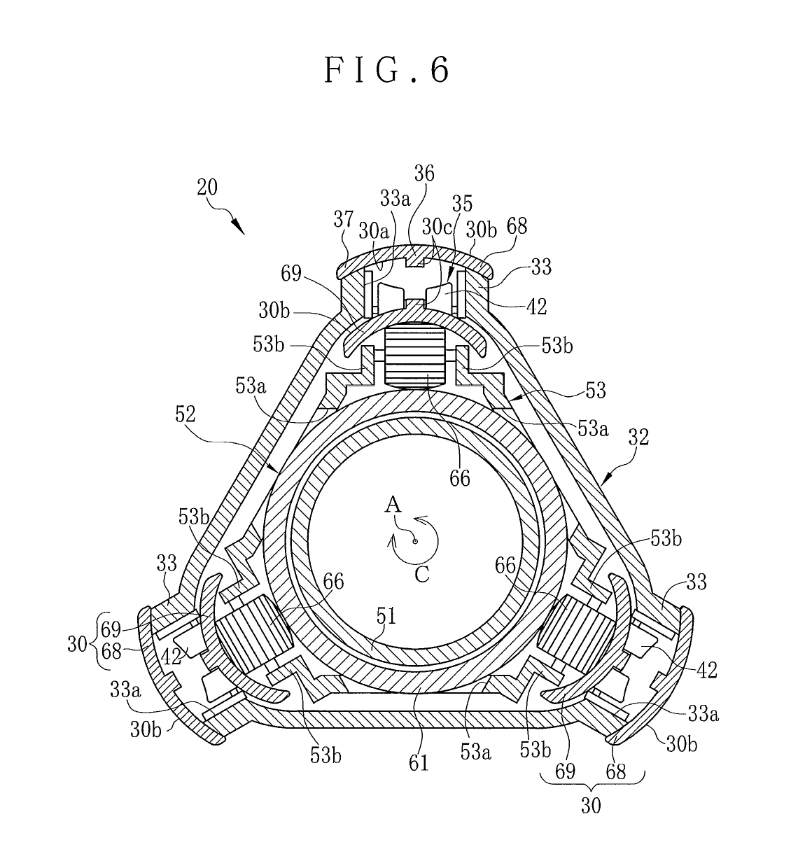

In FIGS. 3, 4, 5 and 6, the housing sleeve 32 has a sleeve form of which a shape as viewed in a cross section is substantially a regular triangular prism with three flat side walls 29, with which three outer support devices or curved walls are combined alternately. The endless belts 30 are wound about the housing sleeve 32. A shaft sleeve 51 is disposed within the housing sleeve 32, and is mounted on the head assembly 11a of the endoscope 10 by receiving insertion.

The endless belts 30 are disposed on respectively outer support devices 33 of the housing sleeve 32. The endless belts 30 are supported on the housing sleeve 32 in an endlessly movable manner. To prepare each of the endless belts 30, a strip of a belt with ends is used at first, and positioned to wrap the housing sleeve 32. Then the ends of the belt are attached to one another by adhesive agent or thermal welding, to obtain the endless belts 30 supported on the housing sleeve 32.

The centrally raised outer support devices 33 of a strip form or pairs of curved walls are formed with the housing sleeve 32, and support an upper belt run 68 of the endless belts 30. The outer support devices 33 have a height increasing from the edge areas to the center area with respect to the transverse direction of the upper belt run 68. Thus, the upper belt run 68 is supported in such an inverted U-shaped curve that its distance from the center of the shaft sleeve 51 decreases from a center portion 36 to lateral side portions 37 of the upper belt run 68. Tensioning devices 34 with an arcuate surface are secured to respectively proximal and distal ends of the outer support devices 33, and contact an inner surface 30a of bent portions of the endless belts 30 for the turn around. The tensioning devices 34 have a semicircular shape. A material for forming the tensioning devices 34 has sufficient smoothness for the endless belts 30 to turn around stably, for example, nylon. Various materials with low friction can be used for the tensioning devices 34, such as PEEK (polyetheretherketone), Teflon (trade name of polytetrafluoroethylene) and the like.

A holder opening 33a is formed in each of the outer support devices 33. A roller device 35 is fitted in the holder opening 33a, and contacts the endless belts 30 in an endlessly movable manner. The roller device 35 includes first, second and third belt rollers 41, 42 and 43 or pressure rollers, and a pair of support plates 40. The belt rollers 41-43 are arranged in the axial direction A. The support plates 40 keep the belt rollers 41-43 rotatable. Note that the belt rollers 41-43 may be directly supported on the housing sleeve 32.

The inner surface 30a of the endless belts 30 contacts the belt rollers 41-43. Corners of the lateral side portions 37 of the endless belts 30 are rounded off. The center portion 36 of the endless belts 30 for contacting the belt rollers 41-43 has a larger thickness than the remaining portion of the endless belts 30, and has a higher rigidity than the remaining portion.

Roller grooves 41a, 42a and 43a are formed in respectively the belt rollers 41-43. A belt ridge 30c is formed to project from the inner surface 30a of the endless belts 30, and extends in the whole length of the endless belts 30. The belt ridge 30c is engaged with the roller grooves 41a, 42a and 43a in a slidable manner, and prevents the endless belts 30 from offsetting in the rotational direction C. Also, a groove 33b is formed in the outer support devices 33 and engaged with the belt ridge 30c in a slidable manner. A groove 34a is formed in the tensioning devices 34 and engaged with the belt ridge 30c in a turnable manner. Note that a coating of lubricant agent is applied to surfaces of the grooves 33b and 34a, the roller grooves 41a-43a and the belt ridge 30c for high smoothness in the contact.

Also, the housing sleeve 32 contains a threaded sleeve 52 as a rotatable drive sleeve for transmission, and a support frame 53 or support sleeve. The threaded sleeve 52 is supported around the shaft sleeve 51 in a rotatable manner. The support frame 53 is disposed around the shaft sleeve 51 and the threaded sleeve 52.

A rear end ring 56 of a hexagonal form is secured to a proximal end of the support frame 53. A front guide ring 57 is fitted on a distal end of the support frame 53 for preventing incidental entry of tissue of a cavity wall of the gastrointestinal tract. A rear guide ring 58 is fitted on the rear end ring 56.

The threaded sleeve 52 supported around the shaft sleeve 51 rotates about an axis extending in the axial direction A. The threaded sleeve 52 has worm gear teeth 61 and spur gear teeth 62. The worm gear teeth 61 are a screw thread of a helical form. The spur gear teeth 62 are disposed at a proximal end of the threaded sleeve 52. A pinion 64 is connected with the coil structure 63, and meshed with the spur gear teeth 62. The pinion 64 is rotated by the coil structure 63, so that the threaded sleeve 52 is rotated by rotation of the spur gear teeth 62.

The support frame 53 as viewed in a cross section is in a hexagonal shape which is defined by removing triangular corner portions smaller than those of the housing sleeve 32. Also, the support frame 53 is positioned in a coaxial manner with the housing sleeve 32. Cutouts 53a are formed in six side walls of the support frame 53. Three of the cutouts 53a are opposed to the outer support devices 33 of the housing sleeve 32. Engagement rollers 66 or drive gears are positioned in the three of the cutouts 53a, and drive the endless belts 30. Among the engagement rollers 66, two are disposed in each one of the cutouts 53a. Pivot brackets 53b or pivot supports are formed on the support frame 53, and support the engagement rollers 66 in a rotatable manner. The engagement rollers 66 are disposed between the first and second belt rollers 41 and 42 and between the second and third belt rollers 42 and 43.

The engagement rollers 66 are meshed with the worm gear teeth 61 of the threaded sleeve 52, and contact the outer surface 30b of the endless belts 30. The engagement rollers 66 have such a form that their tooth surface is curved convexly in a U-shape by following the transverse curve of a lower belt run 69, and are engaged with the lower belt run 69 by tensioning with the belt rollers 41-43 or pressure rollers. The engagement rollers 66 are overlapped with the belt rollers 41-43 in the radial direction of the housing sleeve 32. The lower belt run 69 is curved in a W shape between the belt rollers 41-43 and the engagement rollers 66.

A distal opening 53c is formed in the support frame 53, and receives entry of a distal end of the shaft sleeve 51.

The front guide ring 57 includes an annular ridge 57a and a guide flange 57b. The annular ridge 57a is fitted in the distal opening 53c. The guide flange 57b prevents a cavity wall of the gastrointestinal tract from entangling in the inside of the guide assembly 20. The guide flange 57b has a cup shape with a diameter increasing according to a distance from the annular ridge 57a.

The shape of the guide flange 57b as viewed in a cross section is triangular with three additional outer support devices or curved walls in a manner similar to the housing sleeve 32, but slightly smaller than the housing sleeve 32.

The rear end ring 56 is formed in a hexagonal form similar to the support frame 53. A central opening 56a is defined in the rear end ring 56. A central lumen 51a is defined in the shaft sleeve 51, and communicates with the central opening 56a. A cutout 56b is formed in the rear end ring 56, and contains the pinion 64 in a rotatable manner. The pinion 64 is meshed with the spur gear teeth 62 of the threaded sleeve 52. A hole (not shown) is formed in the rear end ring 56, and receives insertion of the coil structure 63, which is connected to the pinion 64.

The rear guide ring 58 includes an annular ridge 58a and a guide flange 58b in a manner similar to the front guide ring 57. The annular ridge 58a is fitted in the central opening 56a of the rear end ring 56.

The operation of the guide assembly 20 is described now. At first, the head assembly 11a of the elongated tube 11 of the endoscope 10 is entered in the central lumen 51a of the shaft sleeve 51 to mount the guide assembly 20 on the head assembly 11a. Power sources of the processing apparatus, the light source apparatus and the input panel 26 are turned on. The imaging for the diagnosis is ready. Then the head assembly 11a of the endoscope 10 is entered in a gastrointestinal tract of a body of a patient.

The head assembly 11a is advanced to a predetermined site in the body cavity, for example, short of the sigmoid colon. Then the control button 27 of the input panel 26 is depressed to input a command signal for the propulsion. The motor 21 is driven to rotate the coil structure 63 in a predetermined direction. This causes the pinion 64 to rotate. The spur gear teeth 62 rotate to cause the threaded sleeve 52 to rotate.

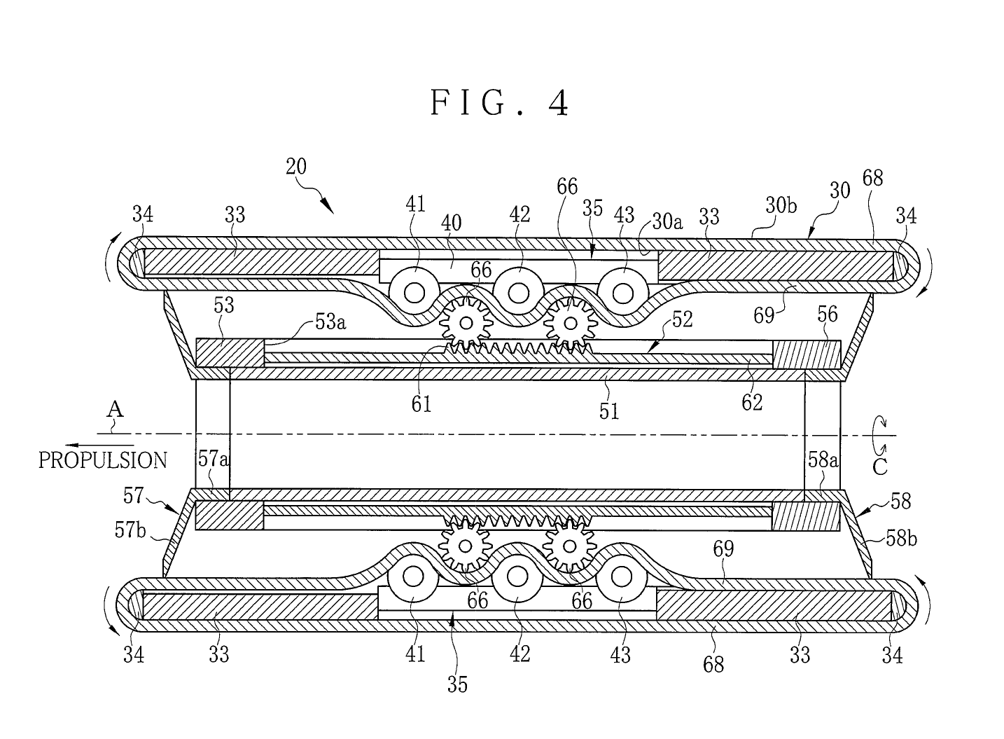

The rotation of the threaded sleeve 52 rotates the engagement rollers 66 or drive gears meshed with the worm gear teeth 61. Thus, the endless belts 30 tensioned between the engagement rollers 66 and the belt rollers 41-43 are turned around in the direction of the arrow in FIG. 4. The outer surface 30b of the upper belt run 68 of the endless belts 30 outside the housing sleeve 32 is moved in the proximal direction in contact with the cavity wall of the body cavity. The outer surface 30b of the lower belt run 69 of the endless belts 30 inside the housing sleeve 32 is moved in the distal direction, so that the endless belts 30 are circulated.

The endless belts 30 contact the cavity wall of the gastrointestinal tract, and are circulated to exert force to move in the proximal direction of the head assembly 11a reverse to the distal direction. The guide assembly 20 applies the force to the body cavity from the distal side toward the proximal side, and propels the head assembly 11a of the endoscope 10 to move in the distal direction in the body cavity. If a doctor or operator wishes to move the guide assembly 20 in the proximal direction, he or she causes the endless belts 30 to operate in the directions reverse to those for the movement in the distal direction.

The endless belts 30 are curved transversely. When the guide assembly 20 propels the head assembly 11a in the body cavity, the lateral side portions 37 can be prevented from contacting a cavity wall of the body cavity. Only the center portion 36 of the endless belts 30 can contact the cavity wall. This is effective in preventing the cavity wall from entanglement on the inner surface 30a of the endless belts 30. The endless belts 30 are disposed at the portions of vertices of the triangular form which may contact the cavity wall the most readily. The endless belts 30 are not disposed on the side walls 29 of the triangular form with considerably low possibility of contacting the cavity wall. The structure is effective in preventing friction against the propulsion, because the side walls 29 without the endless belts 30 are kept from easy contact with the body cavity.

When the speed button 28 of the input panel 26 is operated to input a command signal for change of the speed, rotational speed of the coil structure 63 is changed by controlling the motor 21. Thus, the moving speed of the guide assembly 20 is changed. When the control button 27 of the input panel 26 is operated to input a command signal for return, the coil structure 63 is rotated in a backward direction by controlling the motor 21. Thus, the guide assembly 20 is moved in the proximal direction. When the control button 27 is operated to input a command signal for stop, the coil structure 63 is stopped by stopping the motor 21. Thus, the guide assembly 20 is stopped. It is thus possible to advance the head assembly 11a of the endoscope 10 to a predetermined site in the body cavity as desired by a doctor or operator.

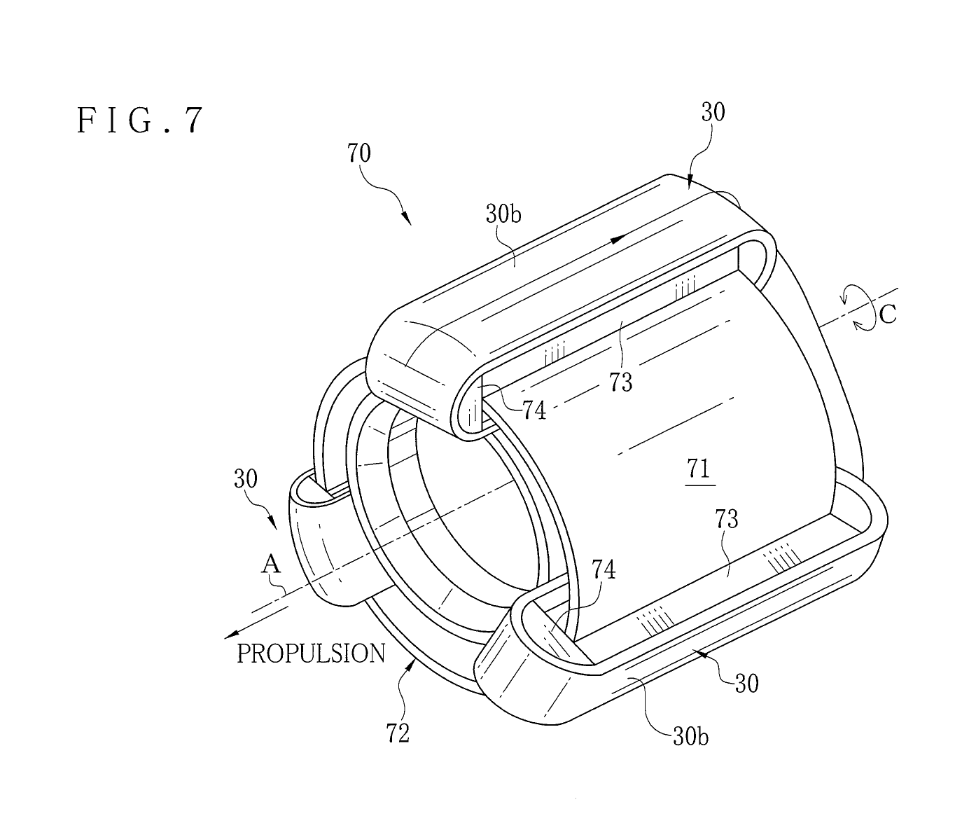

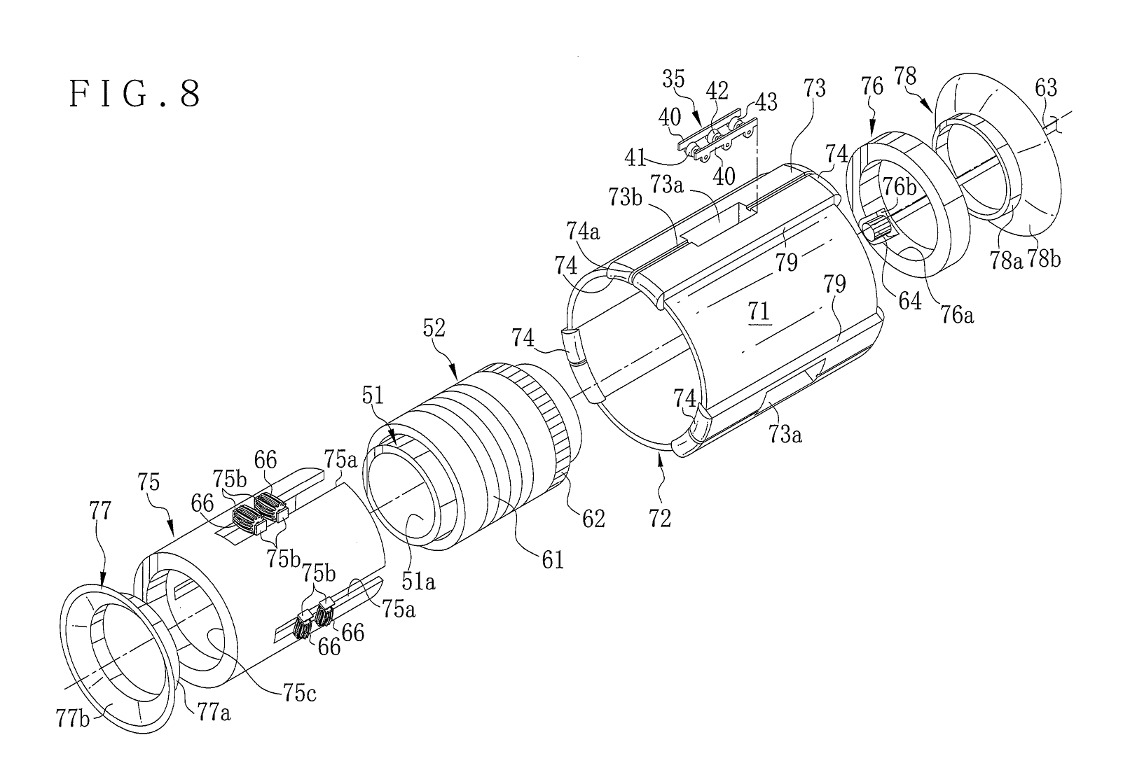

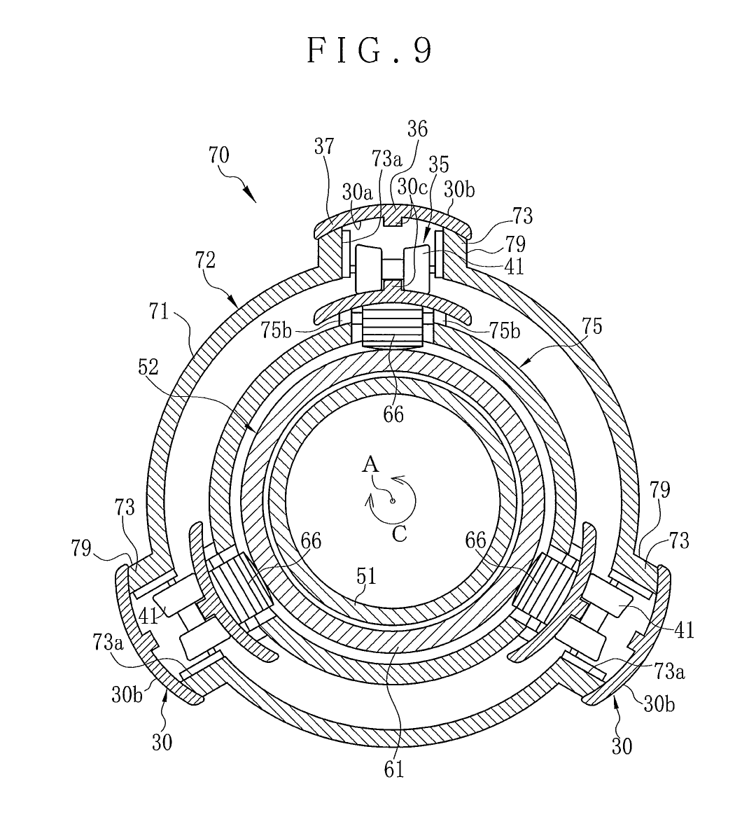

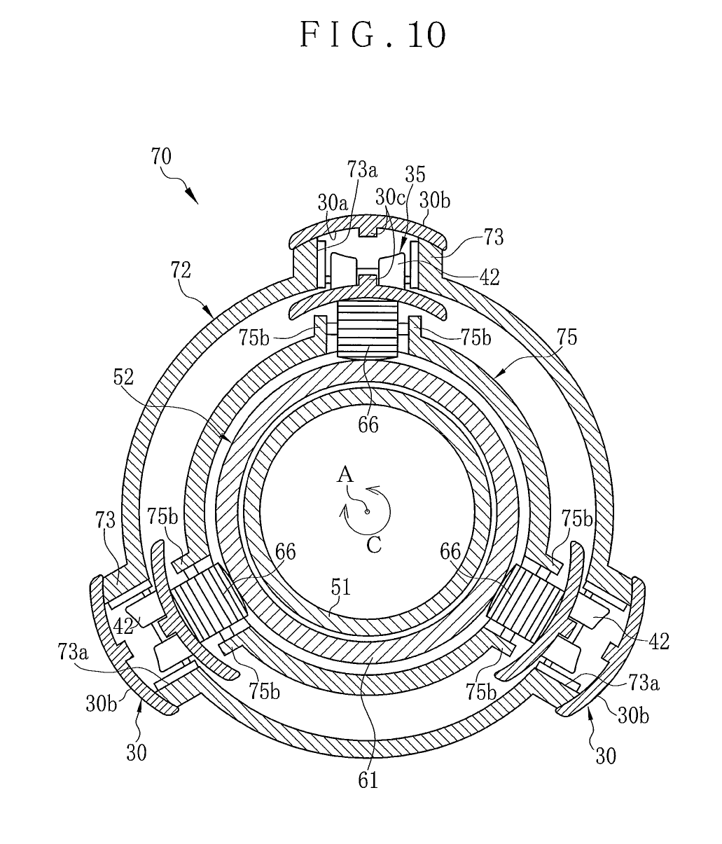

In FIGS. 7-10, a guide assembly 70 of another preferred embodiment is illustrated, and includes a housing sleeve 72 of a cylindrical shape. Elements similar to those of the above embodiment are designated with identical reference numerals.

Centrally raised outer support devices 73 of a strip form or pairs of curved walls are formed on the housing sleeve 72, and support respectively the endless belts 30. The outer support devices 73 have a height increasing from the edge areas to the center area with respect to the transverse direction of the endless belts 30. Thus, the endless belts 30 are supported in such an inverted U-shaped curve that their distance from the center of the shaft sleeve 51 decreases from the center portion 36 to the lateral side portions 37. Tensioning devices 74 with an arcuate surface are secured to respectively proximal and distal ends of the outer support devices 73, have a semicircular shape, and contact the inner surface 30a of bent portions of the endless belts 30.

The housing sleeve 72 includes a cylindrical peripheral surface 71 and an erect surface 79. The erect surface 79 is disposed between the cylindrical peripheral surface 71 and an each edge of the outer support devices 73.

A holder opening 73a is formed in each of the outer support devices 73. The roller device 35 is fitted in the holder opening 73a. A groove 73b is formed in each of the outer support devices 73. The belt ridge 30c of the endless belts 30 is engaged with and received in the groove 73b in a slidable manner. Similarly, a groove 74a is formed in each of the tensioning devices 74 for receiving the belt ridge 30c movably.

A support sleeve 75 or support frame is disposed in the housing sleeve 72, and contains the shaft sleeve 51 and the threaded sleeve 52. The support sleeve 75 is positioned coaxially with the housing sleeve 72.

A rear end ring 76 is secured to a proximal end of the support sleeve 75. A front guide ring 77 is secured to a distal end of the support sleeve 75 for preventing a cavity wall of a body cavity from entanglement in the guide assembly 20. A rear guide ring 78 is secured to the rear end ring 76.

Three cutouts 75a are formed in the support sleeve 75 and opposed to the outer support devices 73 of the housing sleeve 72. Two of the engagement rollers 66 are disposed in each one of the cutouts 75a. Pivot brackets 75b or pivot supports are formed on the support sleeve 75, and support the engagement rollers 66 in a rotatable manner.

A distal opening 75c is formed at a distal end of the support sleeve 75, and receives a distal end of the shaft sleeve 51.

The rear end ring 76 has a central opening 76a and a cutout 76b. The central opening 76a communicates with the central lumen 51a of the shaft sleeve 51. The cutout 76b contains the pinion 64 in a rotatable manner.

The front guide ring 77 includes an annular ridge 77a and a guide flange 77b. The annular ridge 77a is fitted in the distal opening 75c of the support sleeve 75. The guide flange 77b has a cup shape. The rear guide ring 78 includes an annular ridge 78a and a guide flange 78b. The annular ridge 78a is fitted in the central opening 76a of the rear end ring 76. The guide flange 78b has a cup shape.

As the endless belts 30 are curved, the center portion 36 of the endless belts 30 in the transverse direction contacts the cavity wall of the gastrointestinal tract. The lateral side portions 37 will not contact the cavity wall. This is effective in protecting the cavity wall from damages, because there occurs no entanglement of the cavity wall in the area of the inner surface 30a of the endless belts 30.

In the above embodiments, the threaded sleeve and drive sleeve are formed in the triangular and circular shapes as viewed in a cross section. However, those can be formed in other shapes, such as quadrangular, pentagonal, hexagonal and otherwise polygonal shapes.

In the above embodiments, the outer support devices 33, 73 have an arcuate curved surface. However, the outer support devices 33, 73 may have other curved surfaces, such as a surface of a form of an elliptic arc, an arcuate curved surface defined eccentrically with respect to the center of the housing sleeve 32, 72, and the like.

In the above embodiments, the outer support devices 33, 73 have the center area with a larger height than their edge areas. The tensioning devices have the center area with a height equal to that of their edge areas. Alternatively, a height of the outer support devices or curved walls may be decreased gradually toward the tensioning devices of the proximal and distal ends, so that a difference between the center area and the edge areas in the height can be decreased in the axial direction. Also, the tensioning devices may have a center area with a larger height than their edge areas in compliance with the outer support devices of the embodiments.

In the above embodiments, the engagement rollers 66 are rotated by use of the worm gear teeth 61 in the drive sleeve for transmission, so as to drive the endless belts to turn around. However, it is possible for the worm gear teeth 61 or threaded sleeve to drive the endless belts directly without the engagement rollers 66. Note that a rotational direction of the worm gear teeth 61 or threaded sleeve for the propulsion without using the engagement rollers 66 is reverse to that of the worm gear teeth 61 for the propulsion in combination with the engagement rollers 66. A relationship between a rotational direction of the coil structure and the proximal and distal directions for moving the endoscope according to the button panel must be changed suitably for the purpose of the propulsion.

In the above embodiments, the tensioning devices 34 for movably supporting the endless belts are stationary without a rotatable property. However, a pair of rotatable support rollers can be used instead for keeping the endless belts movable endlessly.

In the above embodiments, each of the belt rollers 41-43 has an arcuate form as viewed in a cross section and with a roller surface curved smoothly to retreat. However, the belt rollers 41-43 may be formed in other structures of which a roller diameter decreases at its center portion, for example a form constituted by two conical surfaces in a V form as viewed in a cross section.

In the above embodiments, the belt rollers 41-43 are arranged alternately with the engagement rollers 66 in the axial direction, and are overlapped in the radial direction of the housing sleeve 32, 72. The endless belts 30 can be tensioned in the W shape. The housing sleeve can be positioned exactly. However, it is possible to oppose the engagement rollers 66 to respectively the belt rollers 41-43 in the radial direction of the housing sleeve, which is stationary on the support frame 53. The endless belts 30 can be squeezed tightly between the engagement rollers 66 and the belt rollers 41-43.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide assembly for an endoscope including a section of an elongated tube for entry in a body cavity, comprising:
a shaft sleeve for mounting on said elongated tube;
a housing sleeve disposed around said shaft sleeve;
plural endless belts, secured to said housing sleeve, for endlessly moving in an axial direction of said elongated tube, and propelling said elongated tube by contacting a cavity wall of said body cavity;
a driving device, contained in said housing sleeve, for driving said endless belts;
an outer support device, disposed with said housing sleeve, for supporting said endless belts in a transversely curved state to raise a center portion thereof outwards.

2. A guide assembly as defined in claim 1, further comprising plural belt rollers, secured to said housing sleeve in a rotatable manner, for pressing each of said endless belts for contact with said driving device;
wherein a contact area of said endless belts in contact with said belt rollers has higher rigidity than a remaining area thereof.

3. A guide assembly as defined in claim 2, wherein said belt rollers contact said contact area of said endless belts at a center portion thereof in a transverse direction.

4. A guide assembly as defined in claim 2, wherein each of said endless belts includes:
an upper belt run, disposed outside said housing sleeve, supported by said outer support device, for contacting said cavity wall;
a lower belt run, disposed inside said housing sleeve, to extend between said driving device and said belt rollers in engagement with tension.

5. A guide assembly as defined in claim 4, wherein said driving device includes:
a drive sleeve disposed between said housing sleeve and said shaft sleeve in a rotatable manner;
worm gear teeth of a worm gear formed around said drive sleeve;
an engagement roller, having gear teeth meshed with said worm gear teeth, for turning around said endless belts by engagement therewith.

6. A guide assembly as defined in claim 5, wherein said engagement roller is offset from said belt rollers in said axial direction.

7. A guide assembly as defined in claim 5, wherein said engagement roller has a roller center portion and has a roller diameter increasing toward said roller center portion.

8. A guide assembly as defined in claim 7, wherein said belt rollers have a roller center portion and have a roller diameter decreasing toward said roller center portion.

9. A guide assembly as defined in claim 2, wherein corners of lateral side portions of said endless belts are rounded off.

10. A guide assembly as defined in claim 9, further comprising a belt ridge formed to project from an inner surface of said endless belts.

11. A guide assembly as defined in claim 2, wherein said housing sleeve is in a shape of a polygonal prism, and said outer support device is disposed on each of side edges of said polygonal prism.

12. A guide assembly as defined in claim 11, wherein said outer support device includes a pair of curved walls, arranged along one another, for placement of longitudinal portions of said endless belts.

13. A guide assembly as defined in claim 12, wherein said polygonal prism is a triangular prism.

14. A guide assembly as defined in claim 2, wherein said housing sleeve is cylindrical.

15. A guide assembly as defined in claim 14, wherein said outer support device is constituted by a plurality of outer support devices arranged in an angularly equidistant form around said housing sleeve, and each of said outer support devices includes a pair of curved walls, disposed along one another, for placement of longitudinal portions of said endless belts.
